(19) United States
(12) Patent Application Publication
(10) Pub. No.: US 2022/0118135 A1
(43) Pub. Date: Apr. 21, 2022

(54) AIR AND SURFACE DISINFECTION SYSTEMS AND METHODS

(71) Applicant: HONEYWELL INTERNATIONAL INC., Charlotte, NC (US)

(72) Inventors: Anita Sure, Charlotte, NC (US); Gowtham Kumar Vankayala, Charlotte, NC (US); Harish Nagarajaiah, Charlotte, NC (US); Pramod Kumar T P, Charlotte, NC (US); Tilak Ravi Krishnaswamy, Charlotte, NC (US); Venugopal Krishnappa, Charlotte, NC (US); Krishna Prasad Sabeson, Charlotte, NC (US); Amod Ranade, Charlotte, NC (US)

(21) Appl. No.: 17/450,635

(22) Filed: Oct. 12, 2021

(30) Foreign Application Priority Data
Oct. 16, 2020 (IN) 202011045194

(51) Int. Cl. A61L 2/10; A61L 9/20

(52) U.S. Cl. CPC A61L 2/10; A61L 9/20; A61L 2202/10; A61L 2202/25; A61L 2209/12; A61L 2209/111; A61L 2202/14

(57) ABSTRACT

Devices, methods, and systems for disinfecting air and surfaces are described herein. The systems may include a source of light, a reflector, a motor, and a controller. The source of light may provide ultraviolet germicidal irradiation light and the light may be selectively provided. The reflector may extend along and/or around the source of light. The motor may be coupled to the reflector and cause the reflector to adjust relative to the source of light so as to direct light at different target locations. The controller may determine occupancy in a room in which the system is located. When the room is not occupied, the controller may cause the reflector to be adjusted to an unoccupied position. When the room is occupied, the controller may cause the reflector to be adjusted to an occupied position.

### CROSS-REFERENCE TO RELATED APPLICATIONS

This application claims priority pursuant to 35 U.S.C. 119(a) to India Patent Application No. 202011045194, filed Oct. 16, 2020, which application is incorporated herein by reference in its entirety.

### TECHNICAL FIELD

The present disclosure relates generally to devices, methods, and systems for disinfecting spaces. More particularly, the present disclosure relates to devices, methods, and systems for disinfecting air and/or surfaces of a space.

### BACKGROUND

Disinfection systems are often deployed to disinfect air or surfaces of a space. Air disinfectant systems often includes a forced air mechanism that causes air to pass through or by a disinfecting mechanism. Some disinfection systems utilize a UV light that has germicidal properties and is directed in a primary direction. Such UV light disinfection systems may be directed in a single direction to disinfect air that passes through the UV light rays. Each of the known disinfection systems have advantages and disadvantages.

### SUMMARY

The present disclosure generally relates to disinfection systems and methods that are used to disinfect a space, and more particularly, to systems and methods used to disinfect air and surfaces of a space.

In one example, a disinfection system for a room having a ceiling, one or more walls and a floor may be provided. The illustrative disinfection system includes a housing, a source of ultraviolet (UV) light housed by the housing, a reflector housed by the housing, a motor housed by the housing, and a controller operatively coupled to the motor. The source of UV light may selectively provide a UV light emission (e.g. ON or OFF). The reflector extends partially around the source of UV light and may be oriented relative to the housing so as to direct the UV light emission provided by the source of UV light in a direction that is dependent on the orientation of the reflector relative to the housing. The motor is operatively coupled to the reflector and is configured to change the orientation of the reflector relative to the housing. The controller is configured to receive an indication of occupancy of the room and to control the motor to change the orientation of the reflector relative to the housing between an occupied position and an unoccupied position. When in the occupied position, the reflector directs the UV light emission out of the housing toward one or more of the walls of the room to disinfect air in the room. When in the unoccupied position, the reflector directs the UV light emission out of the housing downward toward the floor of the room to disinfect air in the room and one or more objects in the room.

In another example configuration, a surface and air treatment system may be provided. The illustrative surface and air treatment system includes a source of ultraviolet (UV) light, a reflector extending partially around the source of UV light, a motor in communication with the reflector, an occupancy sensor, and a controller in communication with the source of UV light, the motor, and the occupancy sensor. The controller is configured to initiate the source of UV light and cause the motor to adjust a position of the reflector between an air treatment configuration and a surface treatment configuration based on signals from the occupancy sensor.

In a further example configuration, a method of disinfecting air and surfaces in a space adjacent a source of disinfectant light may be provided. The illustrative method includes positioning a reflector extending partially around the source of disinfectant light in an air treatment configuration, initiating the source of disinfectant light, and determining if an occupant is present in the space adjacent the source of disinfectant light. When it is determined that no occupant is present in the space, the reflector is positioned in a surface treatment configuration. When it is determined that an occupant is present in the space, the reflector is maintained in the air treatment configuration directing disinfectant light into the space.

### BRIEF DESCRIPTION OF THE DRAWINGS

FIG. 1 is a schematic block diagram of an illustrative disinfection system;

FIG. 2 is a schematic block diagram of an illustrative computing device;

FIG. 3 is a schematic diagram of an illustrative disinfection system;

FIG. 4 is a schematic diagram of a portion of an illustrative disinfection system with a reflector in different positions;

FIG. 5 is a schematic diagram of an illustrative disinfection system in a room, where the reflectors are in an unoccupied surface disinfecting position;

FIG. 6 is a schematic diagram of an illustrative disinfection system in a room, where the reflectors are in an occupied air disinfecting position;

FIG. 7 is a flow diagram of an illustrative method for operating a disinfecting system;

FIG. 8 is a flow diagram of an illustrative method for operating a disinfecting system; and

FIG. 9 is a flow diagram of an illustrative method for operating a disinfecting system.

### DESCRIPTION

The present system and approach may incorporate one or more processors, computers, controllers, user interfaces, wireless and/or wire connections, and/or the like, in an implementation described and/or shown.

Buildings and/or facilities often include building automation systems (e.g., heating, ventilation, and air conditioning (HVAC) systems, surveillance systems, security systems, energy management systems, etc.). In some cases, buildings and/or facilities may include cleaning systems and/or cleaning crews that are utilized for cleaning surfaces and/or air of the buildings and/or facilitates. The cleaning systems and/or cleaning crews may be part of the building automation systems and/or may be separate from the building automation systems. Using existing cleaning systems and/or cleaning crews can be tedious, difficult, and sometimes not possible to sufficiently disinfect surfaces and/or air of the buildings and/or facilities over time as needed or required.

This disclosure provides methods and systems for selectively disinfecting air and surfaces in a space or room of a building to reduce risk of infectious disease (e.g., COVID-19, Ebola, influenza, airborne disease, and/or other infectious diseases) transmissions. An illustrative system may be a controlled air and surface (e.g., a dual) disinfectant system utilizing a UV light (e.g., in a UVGI spectrum) emission from a source of UV light and a reflector reflecting the UV light to disinfect air moving upward toward a ceiling and surfaces adjacent to and/or below the source of UV light.

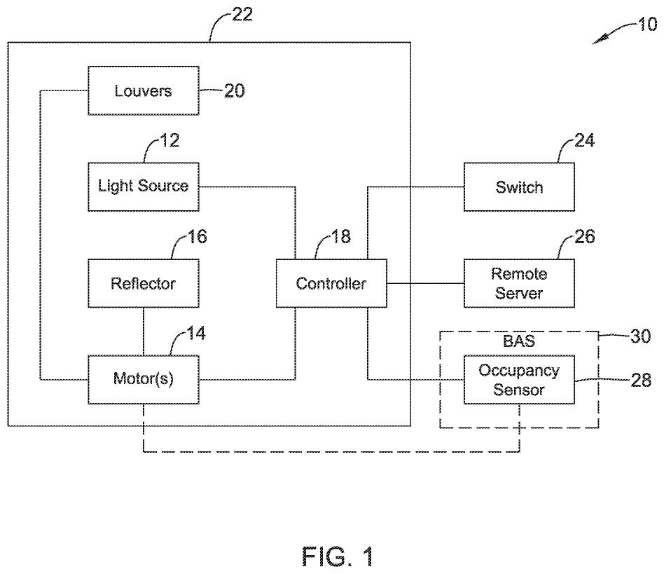

FIG. 1 depicts an illustrative disinfectant or disinfection system 10. The illustrative disinfection system 10 is configured to provide a controlled disinfection of air and surfaces (e.g. dual) in a space adjacent the disinfection system 10.

The illustrative disinfection system 10 includes a source of ultraviolet light 12, a motor(s) 14, a reflector(s) 16 in communication with the motor 14, and a controller 18 in communication with the source of light 12 and the motor 14. In some cases, the disinfection system 10 may include one or more louvers 20 configured to further direct light from the source of light 12 in combination with the reflector 16. In some cases, the disinfection system 10 may include a housing 22 at least partially incorporating and/or housing the source of light 12, the motor 14, the reflector 16, the controller 18, and/or one or more other suitable components of the disinfection system 10. Further, although not required, the disinfection system 10 may include a switch 24 in communication with the controller 18, a remote server 26 in communication with the controller 18, an occupancy sensor 28 in communication with the controller 18, and/or one or more other suitable components. The occupancy sensor 28, as discussed in greater detail below, may be at least partially incorporated in and/or housed in the housing 22 and/or may be part of a building automation system (BAS) 30. Although the disinfection system 10 may be described herein with respect to a single housing 22 and associated components therein and/or a single set of components, the disinfection system 10 may include multiple housings 22 and associated components and/or multiple sets of components or multiple ones of similar components (e.g., multiple sources of light 12, multiple motors 14, multiple reflectors 16, etc.) in a set of components.

The light source 12 may be any suitable light source configured to selectively provide a light having disinfectant qualities. In one example, the light source 12 may be a source of UVGI light (e.g., the light source 12 may provide a UV light emission), but other suitable light having disinfectant qualities is contemplated. In some cases, the UVGI light may provide UVC light with a wavelength in the range of about 100 nanometers (nm) and 280 nm, but this is not required. In some cases, the light source 12 may be configured to provide a plurality of intensities of light emission, which may be controllable by the controller 18.

UVGI light applied to a target may be referred to as a dosage, which is a product of the light intensity and exposure time required to inactivate a particular microorganism. A dosage may be measured in units of microwatt seconds per square centimeter (μW·s/cm²) and/or other suitable units. Different dosages may be required to disinfect areas at which different microorganisms are believed to be present. For example, a dosage of 12,100 μW·s/cm² may be needed for 99.9% disinfection of methicillin resistant Staphylococcus aureus (MRSA), a dosage of 11,500 μW·s/cm² may be needed for 99.9% disinfection of clostridium difficile, and a dosage of 8,400 μW·s/cm² may be needed for a 99.9% disinfection of vancomycin-resistant enterococci.

The disinfection system 10 may include any suitable number of sources of light 12. In some cases, each disinfection system 10 in a room may include a single elongated light source 12 (e.g., a bulb producing light) or more than one light source 12.

The reflector(s) 16 may be any suitable type of reflecting component configured to collect and reflect light from the light source 12 and provide a directional emission in a desired direction. In some cases, the reflector 16 extending at least partially around the light source 12 such that light emitted by the light source 12 may be collected and reflected away from the disinfection system 10 to a desired target region in a room (e.g., a target region of air, a target surface location, etc.). In one example, the reflector 16 may have a cross-sectional shape of a half-circle, a partial-circle, a parabolic shape, and/or other suitable shapes extending at least partially along the light source 12.

The reflector 16 is movable with respect to the light source 12 and/or the housing 22 so as to be able to direct light from the light source 12 in any of a plurality of directions. In one example, the reflector 16 may be configured to have an occupied position and an unoccupied position. The occupied position may position the reflector 16 in a manner that directs light away from locations at which the light may contact people in a space. The unoccupied position may position the reflector 16 in a manner that directs light toward locations of surfaces in the room, such as table surfaces, desk surface, chair surfaces, and counter tops, where such locations could cause light from the light source 12 to undesirably contact people when the space is occupied.

In some cases, the reflector 16 is configured to rotate relative to the housing 22. The reflector 16 may be configured to rotate three hundred sixty (360) degrees relative to the housing 22, one-hundred eighty (180) degrees, one-hundred thirty-five (135) degrees, ninety (90) degrees, forty-five (45) degrees, or any other suitable number of degrees, depending on the application. In one example, when the disinfection system 10 is mounted to a ceiling in a space, the reflector 16 of the disinfection system 10 may be configured to rotate between about zero (0) and about one hundred eighty (180) degrees. When the reflector is in an occupied position (e.g. people are in the space), the reflector may be positioned at 0 or 180 degrees. When the reflector is in an unoccupied position (e.g. people are not detected in the space), the reflector may be positioned at 45 degrees, 90 degrees and 135 degrees. In some cases, when the space is unoccupied, the reflector may rotate across a plurality of positions, such as rotate between zero (0) and one hundred eighty (180) degrees, sometimes multiple times until all target surfaces and/or air has been determined to be disinfected. Sensors and/or calculations based on length of contact with emitted light and a strength of the emitted light may be used to determine when air and/or surfaces have been adequately disinfected. These are just examples.

The reflector 16 may be configured to adjust (e.g., rotate and/or adjust in one or more other suitable manners) relative to the source of light 12. That is, the light source may be stationary relative to the housing, and the reflector 16 may rotate around the source of light 12. Alternatively, the reflector 16 and the source of light 12 may rotate together. In either case, the motor 14 is operatively coupled to the reflector (and sometimes the light source), sometimes through a gear train or the like, to adjust the position of the reflector 16 (and sometimes the source of light 12) relative to the housing 22 in response to one or more received control signals from the controller 18.

In some cases, the disinfection system 10 may include louvers or light baffles 20 that are configured to direct light emitted from the light source 12 into a space. Any suitable number of louvers 20 may be utilized. The louvers 20, when included, may extend along and/or across the light source 12 at a side on which the reflector 16 is not located and may be used to further direct light in concert with the reflector 16.

The louvers 20 may be manually adjusted to a desired position and/or adjusted to the desired position in an automated manner. When adjusted in an automated manner, the louvers may be controlled by the same motor 14 that is utilized to adjust the reflector 16 and/or may be controlled by a different motor 14. Although not required, the louvers 20 may be automatically adjusted to direct light from the light source 12 based on a position of the reflector 16, a direction of movement of the reflector 16, a time during a disinfection cycle, and/or based one or more other suitable factors. In some cases, a plurality of louvers 20 may be radially disposed along the range of motion of the reflector, so that the louvers 20 in the path of the light emission from the reflector 16 are generally parallel with the light emission. Such louvers 20 would allow most of the light emission from the reflector 16 to pass through, but light rays that diverge too much from the desired direction would be blocked by adjacent louvers 20.

The motor 14 may be any suitable type of motor configured to cause position adjustment of the reflector 16. In some case, the motor(s) 14 may be configured to adjust (e.g., rotate) the reflector 16 (and/or the louvers 20) in response to receiving one or more control signals from the controller 18. Example types of motors 14 include, but are not limited to, brushless direct current (DC) motors, brush DC motors, alternating current (AC) squirrel cage motors, AC wound rotor motors, servo motors, stepper motors, and/or other suitable motors.

Any suitable number of motors 14 may be utilized. In one example, a first motor 14 may be operatively coupled to the reflector 16 and utilized to adjust a position of the reflector 16 (e.g., relative to the housing 22), and a second motor 14 may be operatively coupled to the louvers 20, when included, and utilized to adjust a position of the louvers 20 (e.g., relative to the reflector 16, the housing 22, and/or the light source 12).

The occupancy sensor(s) 28 may be any suitable type of sensor configured to determine when a space in which the disinfection system 10 is occupied or becomes occupied (by people or animals). Example types of occupancy sensors 28 may include, but are not limited to, passive infrared (PIR) sensors, temperature sensors, humidity sensors, carbon dioxide (CO₂) sensors, ultrasonic sensors, microwave sensors, video analytics, and/or any other suitable type of sensor configured to detect occupancy of a space.

In some cases, the occupancy sensor 28 may be at least partially located and/or housed by the housing 22 and operatively connected to one or both of the motor 14 and the controller 18. Alternatively, or in addition, the occupancy sensor 28 may be located remote from the housing 22, such on an adjacent wall and operatively coupled to the controller 18. In some cases, the occupancy sensor 28 may be part of a building automation system 30 (e.g., as indicated by the broken line in FIG. 1). For example, the occupancy sensor 28 may be a motion sensor that is part of a security system of a building automation system 30, or a motion sensor that is used to control a zone of a Heating, Ventilation and/or Air Conditioning system of a building automation system 30. These are just examples. When so provided, the building automation system 30 may work in conjunction with the disinfection system 10 of the present disclosure. In the example shown in FIG. 1, the building automation system 30 provides an occupancy signal to the controller 18 of the disinfection system 10. The controller 18 of the disinfection system 10 may be configured to utilize an output of the occupancy sensor 28 of the building automation system 30 to determine when the space in which the disinfection system 10 is located is occupied or not.

When the occupancy sensor 28 detects the space in which the disinfection system 10 is located is occupied, the occupancy sensor 28 may send a signal to one or both of the motor 14 and the controller 18. When a signal indicating the space is occupied is sent to a motor 14, the motor 14 may respond by automatically moving the reflector 16 and/or the louvers 20 to an occupied position and may prevent movement therefrom while the space is occupied. When a signal indicating the space is not occupied is sent to the motor 14, the motor 14 may be caused to operate in accordance with inputted signals from the controller 18 and/or other suitable inputs. That is, the motor logic may act as an interlock. Similarly, when the signal from the occupancy sensor 28 is sent to the controller 18, the controller 18 may respond by automatically moving the reflector 16 and/or the louvers 20 to the occupied position and may prevent movement therefrom while the space is occupied. When a signal indicating the space is not occupied is sent to the controller 18, the controller 18 may cause the motor 14 to adjust the reflectors 16 and/or the louvers 20 according to a control methods for disinfecting the space utilizing unoccupied positions of the reflectors 16 and/or the louvers 20.

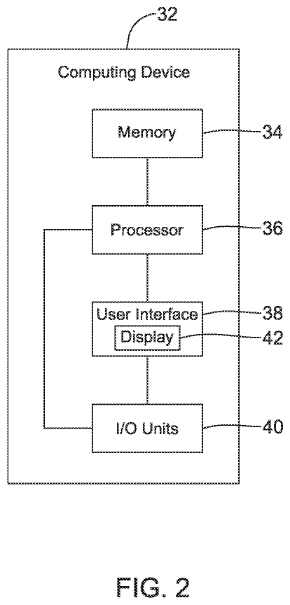

The controller 18 may be any suitable type of controller configured to at least partially control the operation of the motor(s) 14 and/or the light source 12 to operate and/or actuate functionality of the disinfection system 10. In some cases, the controller 18 may be one or more computing devices (e.g., as discussed with respect to FIG. 2 and/or other suitable computing devices). The controller 18 may be configured to receive an indication of occupancy of the space in which the disinfection system 10 is located and control the motor 14 and/or the light source 12 based on the indication of the occupancy. The indication of occupancy may be received from the occupancy sensor 28 and/or one or more other suitable components. Further, and in some cases, the controller 18 may be configured to at least partially control the operation of the motors 14 and/or the light sources 12 based, at least in part, on input from other components of the BAS 30 and/or other components of the disinfecting system 10.

In some cases, the controller 18 may be operatively coupled to a switch 24 that is configured to initiate, stop, and/or adjust functionality (e.g., initiate or stop operation of the light source 12, movement of the motor 14, etc.) of the disinfection system 10. The switch(es) 24 may be located on, in, or at the housing 22 and/or at a remote location. Example remote locations for the switch 24 may include, but are not limited to, locations on walls in the space the disinfection system 10 is configured to disinfect, a location at a space adjacent to the space the disinfection system 10 is configured is configured to disinfect, a location in a building or facility control room, a location of the remote server 26, a location of an application on a mobile device, and/or one or more other locations remote from the housing 22 of the disinfection system 10.

The switch 24 may be configured to turn the disinfection system 10 and/or components thereof on and/or off. Additionally, and/or alternatively, the switch 24 may include further features that may be used to set a strength of the light emitted from the light source 12 (e.g. like a dimmer), a position of the reflector 16, a disinfecting cycle, a disinfecting schedule, etc.

The remote server 26, when provided, may be any suitable type of computing device located remote from the space in which the disinfection system 10 is configured to disinfect. The remote server 26 may be in operative communication with the controller 18 over one or more wired or wireless connections. The remote server 26 may be utilized to coordinate operations of the disinfection system 10 with operations of other components, such as one or more components of the BAS 30, and/or to provide data to a central location at which data may be stored and/or from which the data may be distributed to one or more various user interfaces from which a control action may or may not be taken (e.g., manually or automatically).

The remote server 26, other suitable components of the BAS 30, other suitable components of the disinfecting system 10, user mobile devices, user computing devices, and/or other suitable computing devices may communicate over one or more wired and/or wireless networks. Example networks include, but are not limited to, a distributed computing environment (e.g., a cloud computing environment), a wide area network (WAN) (e.g., the Internet), a local area network (LAN), a personal area network (PAN), a campus area network (CAN), or metropolitan area network (MAN), among other types of network relationships. The communications over the network(s) and between computing devices may traverse through a firewall, but this is not required. Further, one or more gateway devices may be utilized to facilitate communication over the one or more networks.

FIG. 2 illustrates an example of a computing device 32 that may be or may be included in the computing devices discussed herein. The computing device 32 may represent all or part of one or more of the light source 12, the motor 14, the controller 18, the switch 24, the remote server 26, the occupancy sensor 28, components of the BAS 30, and/or one or more other computing device components. Although not shown, the computing device 32 may include a clock and/or a timer. The computing device 32 may be and/or may be part of, for instance, a smart phone, a tablet, a personal digital assistant (PDA), a personal computer, a motor, a network device, a light source, a controller, a sensor, a switch, a remove server, and/or other suitable computing device. However, configurations of the present disclosure are not limited to a particular type of computing device 32. In some cases, the computing device 32 may include memory 34, one or more processors 36, one or more user interfaces 38, one or more input/output (I/O) units 40, and/or one or more other suitable computing components.

As shown in FIG. 2, the computing device 32 may include the memory 34 and a processor 36 that may communicate with one another such that the processor 36 may execute instructions (e.g., application program code of a mobile application or software, control algorithm software, and/or other suitable instructions) stored on the memory 34. The computing device 32 may further include a user interface 38, an I/O unit 40, and/or one or more other suitable components.

The memory 34 may be any type of storage medium that can be accessed by the processor 36 to perform various examples of the present disclosure. For example, the memory 34 may be a non-transitory computer readable medium having computer readable instructions (e.g., computer or application program instructions) stored thereon that are executable by the processor 36 for performing one or more methods described herein.

The memory 34 may be may be volatile or nonvolatile memory. The memory 34 may also be removable (e.g., portable) memory, or non-removable (e.g., internal) memory. For example, the memory 34 may be random access memory (RAM) (e.g., dynamic random access memory (DRAM) and/or phase change random access memory (PCRAM)), read-only memory (ROM) (e.g., electrically erasable programmable read-only memory (EEPROM) and/or compact-disk read-only memory (CD-ROM)), flash memory, a laser disk, a digital versatile disk (DVD) or other optical disk storage, and/or a magnetic medium such as magnetic cassettes, tapes, or disks, among other types of memory.

Further, although the memory 34 is illustrated as being located in the computing device 32, embodiments of the present disclosure are not so limited. For example, the memory 34 may also be located internal to another computing resource (e.g., enabling computer readable instructions to be downloaded over the Internet or another wired or wireless connection).

The processor 36 of the computing device 32 may include a single processor or more than one processor working individually or with one another (e.g., dual-core, etc.). The processor 36 may be configured to execute instructions, including instructions that may be loaded into the memory 34 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices.

The user interface 38, when provided, may be any suitable user interface and/or user interface components configured to facilitate a user of the computing device 32 interacting with the computing device 32 via the user interface 38. For example, the user interface 38 may be used to provide information to and receive information from the user of the computing device 32. For instance, the user interface 38 may receive selections of dates and times for performing a disinfection cycle using the disinfection system 10. The user interface 38 may include a keyboard or keyboard functionality, a pointer (e.g., a mouse, touch pad, or touch ball) or pointer functionality, a microphone, a speaker, a light system, a haptic system, a camera, a video camera, and/or other suitable user interface features the user may use to input information into and/or receive information from the computing device 32. Configurations of the present disclosure, however, are not limited to a particular type(s) of user interface 38.

In some cases, the user interface 38 may include a graphical user interface (GUI) that may have a display 42 (e.g., a screen) that may provide and/or receive information to and/or from the user of the computing device 32. The display 42 may be, for instance, a touch-screen (e.g., the GUI may include touch-screen capabilities).

The I/O unit 40 may be and/or include any type of communication port(s) and may facilitate wired and/or wireless communication with one or more networks. In one example, the I/O unit 40 may facilitate communication with one or more networks and/or other devices through any suitable connection including, but not limited to, radio communication, Ethernet, cellular communication, ZigBee, REDLINK™, Bluetooth, Bluetooth Low Energy (BLE), WiFi, IrDA, dedicated short range communication (DSRC), EnOcean, Near Field Communication (NFC), and/or any other suitable common or proprietary wired or wireless protocol. In one example, the I/O unit 40 may at least include a port configured to communicate over a Bluetooth connection with one or more components of or in communication with the BAS 30 and/or the disinfection system 10.

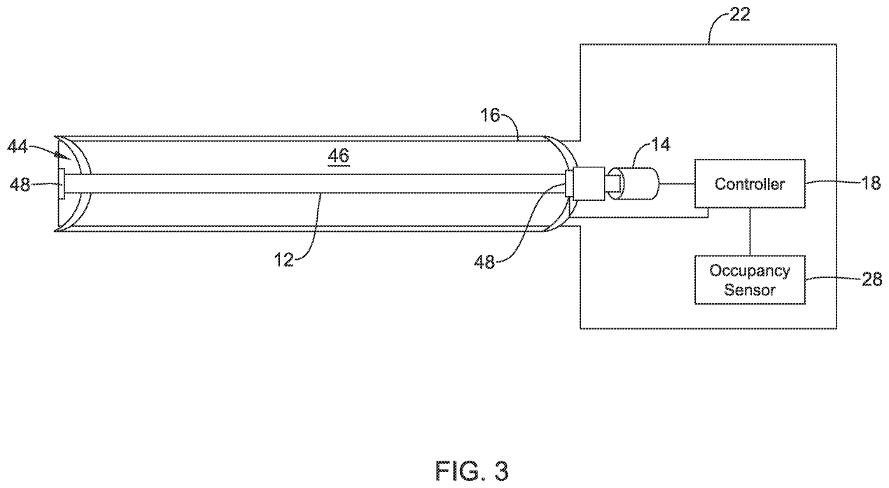

FIG. 3 is a schematic diagram of an illustrative disinfection system 10. The illustrative disinfection system 10 depicted in FIG. 3 includes the reflector 16 extending at least partially along and partially around the source of light 12 (e.g., the source of UV light). As shown in FIG. 3, the reflector 16 may have a cross-sectional shape of a half-circle, partial ellipse, parabola, or similar shape, such that light emitted by the light source 12 may be collected and reflected in a desired direction.

The housing 22 depicted in FIG. 3 may house the motor 14, the controller 18, and the occupancy sensor 28. Other suitable components of the disinfecting system 10 may be housed by the housing 22 including, but not limited to, the light source 12, the reflector 16, and/or other components of the disinfecting system 10. As detailed herein, in some cases, the occupancy sensor 28 may be located outside of and may be remote from the housing 22.

In the example shown, the controller 18 is in communication with the light source 12, the motor 14, and the occupancy sensor 28. In operation, the controller 18 may be configured to control operation of the light source 12 and the motor 14 to disinfect air and/or surfaces of a room and/or space adjacent the disinfection system 10. For example, the controller 18 may send signals to the motor 14 to cause the motor 14 to rotate and adjust a position of the reflector 16 relative to the housing 22 (and thus the room). Further, the controller 18 may send a signal to the light source 12, which may be secured in a stationary manner (e.g., within a socket 48 and/or otherwise secured in a suitable manner) relative to the housing 22, to cause the light source 12 to emit light, stop emitting light and/or control an intensity of the emitted light.

The occupancy sensor 28 may provide data of and/or related to occupancy of the space and/or room having air and surfaces to be disinfected. Although other computing devices may determine whether the occupancy sensor 28 has sensed an occupant in a space, the controller 18 may be configured to use signals from the occupancy sensor 28 to determine whether the occupancy sensor has sensed an occupant in the space. In some cases, the controller 18 may initiate the source of light 12 and cause the motor 14 to adjust a position of the reflector 16 between an air treatment configuration (e.g., an occupied configuration or position) and a surface treatment configuration (e.g., an unoccupied configuration or position) based on signals from the occupancy sensor 28.

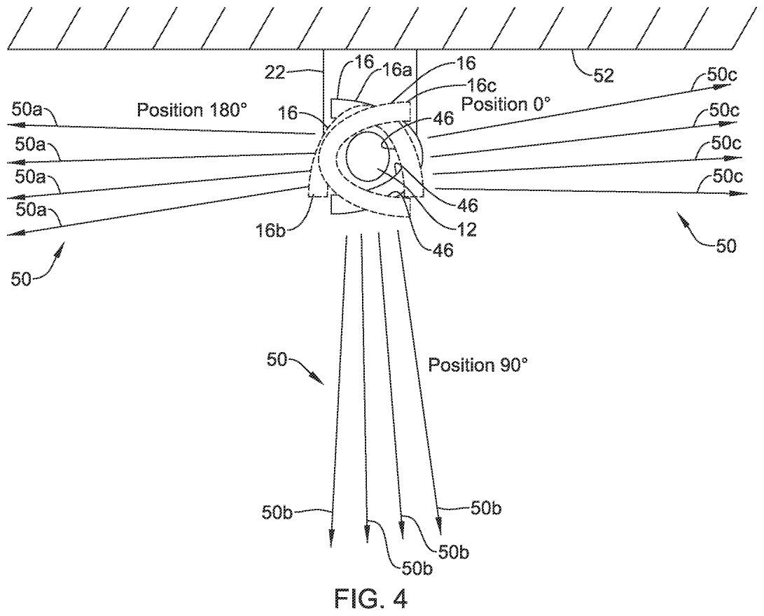

FIG. 4 depicts a plan view of an end of the light source 12 emitting light 50, the reflector 16 reflecting the light 50 off of the inner surface 46, and the housing 22 mounted to a ceiling 52 of a room, where the reflector 16 is depicted in three different positions (e.g., represented by reflectors 16a , 16b , 16c transposed on one another). The first position of the reflector 16 is represented by the reflector 16a at the one hundred eight (180) degree position (e.g., an occupied or air treatment configuration) at which light rays 50a may be emitted in a direction generally parallel to a surface of the ceiling 52. Generally parallel is considered to be plus or minus 15 degrees from parallel or less). The second position of the reflector 16 is represented by the reflector 16b at the ninety (90) degree position (e.g., an unoccupied or surface treatment configuration) at which light rays 50b are emitted in a direction generally perpendicular to or away from the ceiling 52. The third position of the reflector 16 is represented by the reflector 16c at the zero (0) degree position (e.g., an occupied or air treatment configuration) at which light rays 50c are emitted in a direction generally parallel to the surface of the ceiling 52.

Although the reflector 16 is depicted in only three positions between zero (0) degrees and one hundred eighty (180) degrees, the reflector 16 may be moved to any other positions between zero (0) and one hundred eight (180) degrees or other suitable range as desired. In some cases, the motor 14 may cause the reflector 16 to move between zero (0) and one hundred eighty (180) degrees at a controlled rate while the light source 12 is emitting light 50 to disinfect air and/or surfaces of the space adjacent the disinfection system when the room is unoccupied. In some cases, when the room is unoccupied, the reflector 16 may be driven to span at least forty-five (45) degrees, at least ninety (90) degrees, at least one hundred seventy (170) degrees, and/or one or more other suitable ranges.

As can be seen in FIG. 4, the reflectors 16 (e.g., the reflective surfaces 46 of the reflectors) may have a parabolic shape. Such a configuration may have beneficial angles of reflections toward a target location. Even so, other suitable configurations of the reflective surfaces 46 of the reflectors is contemplated.

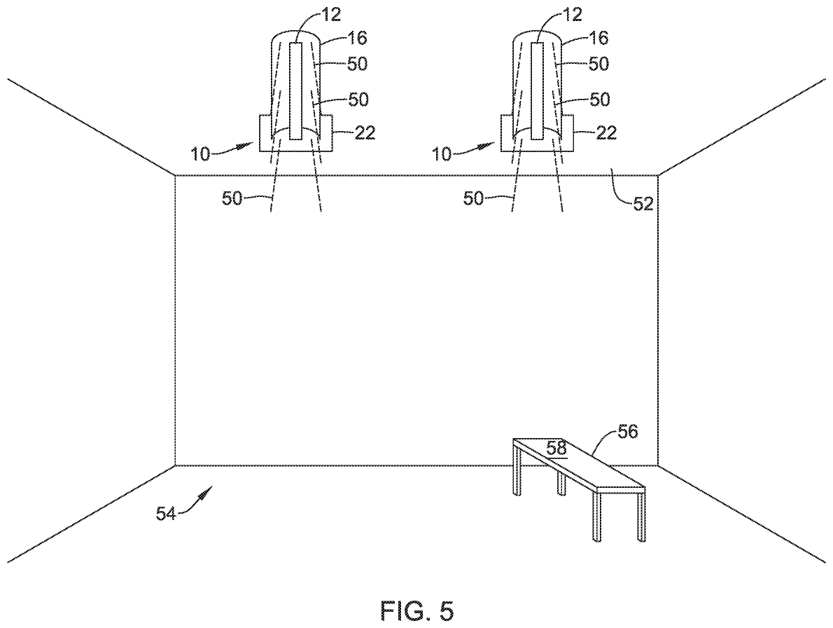

FIG. 5 is a schematic diagram depicting illustrative disinfection systems 10 in a room 54 having a ceiling and walls extending downward from the ceiling to a floor, where the housings 22 of the disinfection systems 10 is secured to the ceiling 52. The reflectors 16 of the disinfection systems 10 depicted in FIG. 5 are in an unoccupied position (or a surface treatment configuration) that is configured to reflect the light 50 from the light sources 12 toward surfaces 58 of one or more objects 56 in the room 54.

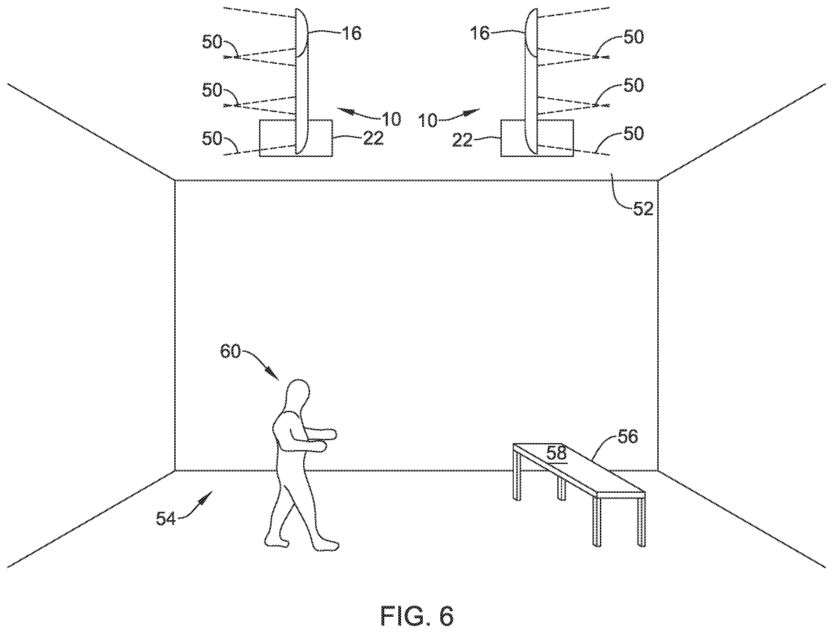

FIG. 6 is a schematic diagram depicting illustrative disinfection systems 10 in the room 54, where the housings 22 are secured to the ceiling 52. The reflectors 16 of the disinfection systems 10 depicted in FIG. 6 are in an occupied position (or an air treatment configuration) that is configured to reflect the light 50 from the light sources 12 in a direction generally parallel to the ceiling 52. In some cases, the reflectors 16 may be adjusted to and/or maintained in the occupied position (or the air treatment configuration) in response to detection of an occupant 60 in the room 54 (e.g., detection by the occupancy sensor 28 and/or other suitable occupancy determiner).

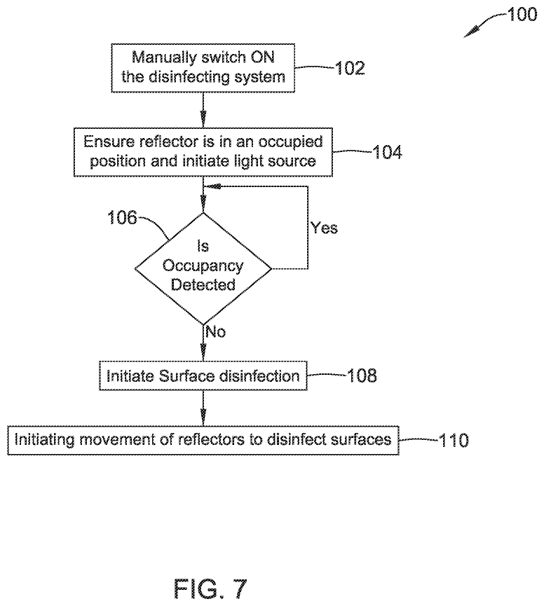

FIG. 7 depicts a flow diagram showing an illustrative method 100 of disinfecting air and surfaces of a space (e.g., the room 54 and/or other suitable space). The method 100 may be manually initiated and a user may manually switch ON (e.g., using the switch 24 and/or other suitable switch or initiator) 102 a disinfecting system (e.g., the disinfecting system 10 and/or other suitable disinfecting system). In response to switching ON the disinfecting system, the disinfecting system automatically ensured 104 a reflector (e.g., the reflector 16 and/or one or more other suitable reflectors) is in an occupied position (air disinfection position), and then initiates the light source (e.g., the light source 12 and/or other suitable light source). In one example, a controller (e.g., the controller 18 and/or other suitable controller) may receive a signal in response to switching 102 a switch ON, and the signal may cause the controller to ensure the reflector is in the occupied position (air disinfection position), and then initiate the light source. If it is determined that the reflector is not in the occupied position (air disinfection position) when the disinfecting system is switched on, the disinfection system automatically adjust the reflector to the occupied position (air disinfection position) before switching on the light source.

The method 100 may include determining 106 if occupancy of the room has been detected. The controller may determine whether the room is occupied based on feedback from an occupancy sensor (e.g., the occupancy sensor 28 and/or other suitable occupancy sensor). Alternatively, or additionally, the controller may receive a signal from a BAS (e.g., the BAS 30 and/or other suitable BAS) central command (e.g., a remote server connected to the BAS, a central BAS workstation for a building, etc.) indicating the room is occupied and determine whether the room is occupied based on whether the signal has been received. Further, determining 106 if occupancy of the room has been detected may be continuously repeated while the disinfecting system has been switched ON, may be repeated at predetermined time periods (e.g., at an occupancy sensor sampling rate, etc.), repeated in response to a predetermined event (e.g., new data crossing a threshold, etc.), and/or repeated or not at one or more other suitable times.

If the room is determined to be occupied, the disinfecting system maintains the reflector in the occupied position (air disinfection position). If the room is determined to be unoccupied, the disinfecting system may automatically initiate 108 surface disinfection. Initiating surface disinfection may include adjusting the reflector to one or more surface disinfecting positions (e.g., positions between zero (0) and one hundred eighty (180) degrees) to cause light emitted from the light source and reflected from the reflector to disinfect surfaces of objects or features in the room. In response to the initiation 108 of surface disinfection, the method 100 may include automatically initiating 110 movement of reflectors to cause reflected light to contact surfaces of objects and features in the room in a manner that facilitates disinfecting those surfaces.

The movement of the reflectors to disinfect surfaces of the room in the method 100 may be an automatic gradual and/or continuous movement based on algorithms in the controller of the disinfecting system that are configured to determine when a surface has been sufficiently disinfected and/or other suitable algorithms. Alternatively, the movement of the reflectors to disinfect surfaces of the room may be an automatic discreet or step movement configured to adjust the reflectors to predetermined positions between a range of motion of the reflectors for a predetermined amount of time.

In some cases, when the user manually switches ON (e.g., using the switch 24 and/or other suitable switch or initiator) 102 the disinfecting system, the light source may remain OFF until it is determined that the room is unoccupied (e.g. via an occupancy sensor). That is, if the room is occupied when the user manually switches ON (e.g., using the switch 24 and/or other suitable switch or initiator) 102 the disinfecting system, the light source will remain OFF until the room becomes unoccupied.

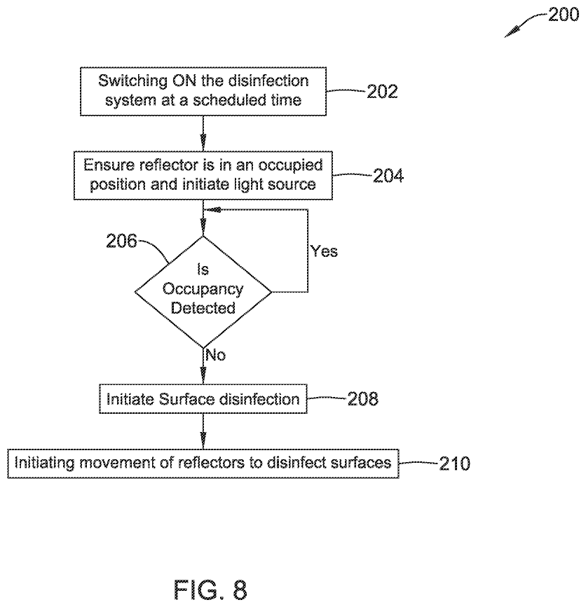

FIG. 8 depicts a flow diagram showing another illustrative method 200 of disinfecting air and surfaces of a space (e.g., the room 54 and/or other suitable space). The method 200 may be initiated according to an established schedule and a disinfecting system (e.g., the disinfecting system 10 and/or other suitable disinfecting system) may be automatically switched ON 202 at a scheduled time according to the established schedule. The schedule may be saved in memory (e.g., the memory 34 of the disinfecting system 10 and/or other suitable memory).

In response to the disinfecting system automatically switching ON according to the schedule, the disinfecting system may automatically ensure 204 a reflector (e.g., the reflector 16 and/or one or more other suitable reflectors) is in an occupied position (air disinfection position) and initiate the light source (e.g., the light source 12 and/or other suitable light source). In one example, a controller (e.g., the controller 18 and/or other suitable controller) may receive a signal in response to the disinfecting system switching 202 ON and the signal may cause the controller to initiate the light source and/or ensure the reflector is in the occupied position (air disinfection position) position. If it is determined the reflector is not in the occupied position (air disinfection position) when the disinfecting system is switched on according to the schedule, the disinfection system automatically adjust the reflector to the occupied position (air disinfection position).

The method 200 may include determining 206 if occupancy of the room has been detected. The controller may determine whether the room is occupied based on feedback from an occupancy sensor (e.g., the occupancy sensor 28 and/or other suitable occupancy sensor). Alternatively, or additionally, the controller may receive a signal from a BAS (e.g., the BAS 30 and/or other suitable BAS) central command (e.g., a remote server connected to the BAS, a central BAS workstation for a building, etc.) indicating the room is occupied and determine whether the room is occupied based on whether the signal has been received. Further, determining 206 if occupancy of the room has been detected may be continuously repeated while the disinfecting system has been switched ON, may be repeated at predetermined time periods (e.g., at an occupancy sensor sampling rate, etc.), repeated in response to a predetermined event (e.g., new data crossing a threshold, etc.), and/or repeated or not at one or more other suitable times.

If the room is determined to be occupied, the disinfecting system may maintain the reflector in the occupied position (air disinfection position). If the room is determined to be unoccupied, the disinfecting system may automatically initiate 208 surface disinfection if needed. Initiating surface disinfection may include adjusting the reflector to unoccupied surface disinfecting positions (e.g., positions between zero (0) and one hundred eighty (180) degrees) to cause light emitted from the light source to be directed into the room to disinfect surfaces of objects or features in the room. In response to the initiation 208 of surface disinfection, the method 200 may include automatically initiating 210 movement of reflectors to cause reflected light to contact surfaces of objects and features in the room in a manner that facilitates disinfecting those surfaces.

The movement of the reflectors to disinfect surfaces of the room in the method 200 may be an automatic gradual and/or continuous movement based on algorithms in the controller of the disinfecting system that are configured to determine when a surface has been sufficiently disinfected and/or other suitable algorithms. Alternatively, the movement of the reflectors to disinfect surfaces of the room may be an automatic discreet or step movement configured to adjust the reflectors to predetermined positions between a range of motion of the reflectors for a predetermined amount of time.

In some cases, when the disinfecting system is automatically switched ON 202 at a scheduled time, the light source may remain OFF until it is determined that the room is unoccupied (e.g. via an occupancy sensor). That is, if the room is occupied when the disinfecting system is automatically switched ON 202 at a scheduled time, the light source will remain OFF until the room becomes unoccupied.

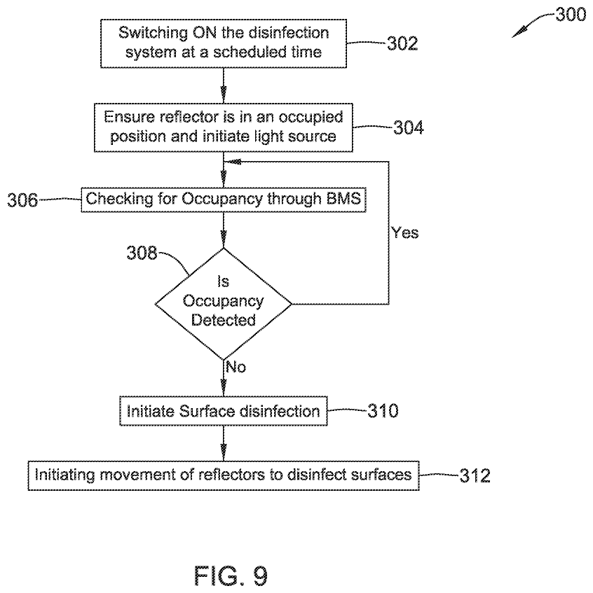

FIG. 9 depicts a flow diagram showing another illustrative method 300 of disinfecting air and surfaces of a space (e.g., the room 54 and/or other suitable space). The method 300 may be initiated according to an established schedule, and a disinfecting system (e.g., the disinfecting system 10 and/or other suitable disinfecting system) may be automatically switched ON 302 at a scheduled time according to the established schedule. The schedule may be saved in memory (e.g., the memory 34 of the disinfecting system and/or other suitable memory). Alternatively, or additionally, the method 300 may be manually initiated and a user may manually switch ON (e.g., using the switch 24 and/or other suitable switch or initiator) the disinfecting system.

In response to the disinfecting system automatically switching ON according to the schedule and/or being manually switched ON, the disinfecting system may automatically ensure 304 a reflector (e.g., the reflector 16 and/or one or more other suitable reflectors) is in an occupied position (air disinfection position) and initiate the light source (e.g., the light source 12 and/or other suitable light source). In one example, a controller (e.g., the controller 18 and/or other suitable controller) may receive a signal in response to the disinfecting system switching 102 ON and the signal may cause the controller to initiate the light source and/or ensure the reflector is in an occupied position (air disinfection position). If it is determined the reflector is not in an occupied position (air disinfection position) when the disinfecting system is switched on, the disinfection system may automatically adjust the reflector to an occupied position (air disinfection position).

The method 300 may include proactively checking 306 with BAS (e.g., the BAS 30 and/or other suitable BAS) of a building or facility in which the room or space is located that the disinfecting system is configured to disinfect as to whether the room or space is occupied. The BAS may be able to determine occupancy from one or more dedicated occupancy sensors and/or other suitable sensors or devices that may provide data or information related to occupancy (e.g., cameras, temperature sensors, humidity sensors, particle sensors, pollutant detectors, etc.). Once feedback is received from the BAS, a controller (e.g., the controller 18 and/or other suitable controller) of the disinfecting system may determine 308 if occupancy of the room has been detected. Further, checking 306 occupancy with the BAS and/or determining 308 if occupancy of the room has been detected may be continuously repeated while the disinfecting system has been switched ON, may be repeated at predetermined time periods (e.g., at an occupancy sensor sampling rate, etc.), repeated in response to a predetermined event (e.g., new data crossing a threshold, etc.), and/or repeated or not at one or more other suitable times.

If the room is determined to be occupied, the disinfecting system may maintain the reflector in the occupied position (air disinfection position). If the room is determined to be unoccupied, the disinfecting system may automatically initiate 310 surface disinfection when needed. Initiating surface disinfection may include adjusting the reflector to unoccupied surface disinfecting positions (e.g., positions between zero (0) and one hundred eighty (180) degrees) to cause light reflected from the reflector to disinfect surfaces of objects or features in the room. In response to the initiation 310 of surface disinfection, the method 300 may include automatically initiating 312 movement of reflectors to cause reflected light to contact surfaces of objects and features in the room in a manner that facilitates disinfecting those surfaces.

The movement of the reflectors to disinfect surfaces of the room in the method 300 may be an automatic gradual and/or continuous movement based on algorithms in the controller of the disinfecting system that are configured to determine when a surface has been disinfected and/or other suitable algorithms. Alternatively, the movement of the reflectors to disinfect surfaces of the room may be an automatic discreet or step movement configured to adjust the reflectors to predetermined positions between a range of motion of the reflectors for a predetermined amount of time.

In some cases, when the disinfecting system is automatically switched ON 302 at a scheduled time, the light source may remain OFF until it is determined that the room is unoccupied (e.g. via an occupancy sensor). That is, if the room is occupied when the disinfecting system is automatically switched ON 302 at a scheduled time, the light source will remain OFF until the room becomes unoccupied.

Although FIGS. 7-9 depict various methods for initiating and operating a disinfecting system, others are contemplated. For example, in some cases, the source of light may be initiated in response to the BAS detecting pollutants in the space adjacent the source of disinfectant light based on sensed air quality and/or detected pollutants in the space. In another example, occupancy may be detected by detecting one or more triggers in the space or building (e.g., lights turned on, temperature adjusted, keyboard or mouse action on a computer, etc.)

Although specific embodiments have been illustrated and described herein, those of ordinary skill in the art will appreciate that any arrangement calculated to achieve the same methods can be substituted for the specific embodiments shown. This disclosure is intended to cover any and all adaptations or variations of various embodiments of the disclosure.

It is to be understood that the above description has been made in an illustrative fashion, and not a restrictive one. Combination of the above embodiments, and other embodiments not specifically described herein will be apparent to those of skill in the art upon reviewing the above description.

The scope of the various embodiments of the disclosure includes any other applications in which the above structures and methods are used. In the foregoing Description, various features are grouped together in example embodiments illustrated in the figures for the purpose of streamlining the disclosure. This method of disclosure is not to be interpreted as reflecting an intention that the embodiments of the disclosure require more features than are expressly recited in each claim.

What is claimed is:

1. A disinfection system for a room having a ceiling, one or more walls and a floor, the disinfection system comprising:
a housing;
a source of ultraviolet (UV) light housed by the housing, the source of UV light selectively providing a UV light emission;
a reflector housed by the housing, the reflector extending partially around the source of UV light and oriented relative to the housing so as to direct the UV light emission provided by the source of UV light in a direction that is dependent on the orientation of the reflector relative to the housing;
a motor housed by the housing, the motor operatively coupled to the reflector and configured to change the orientation of the reflector relative to the housing;
a controller operatively coupled to the motor, the controller configured to receive an indication of occupancy of the room and to control the motor based on the indication of occupancy of the room to change the orientation of the reflector relative to the housing between an occupied position and an unoccupied position;
wherein in the occupied position, the reflector directs the UV light emission out of the housing toward one or more of the walls of the room to disinfect air in the room; and
wherein in the unoccupied position, the reflector directs the UV light emission out of the housing downward toward the floor of the room to disinfect air in the room and one or more objects in the room.

2. The disinfection system of claim 1, wherein the housing is configured to be mounted at or near the ceiling of the room, wherein in the occupied position, the reflector directs the UV light emission out of the housing toward one or more of the walls of the room to disinfect upper air in the room.

3. The disinfection system of claim 2, wherein the upper air in the room is circulated by convection in the room.

4. The disinfection system of claim 2, wherein in the unoccupied position, the reflector directs the UV light emission out of the housing downward toward the floor of the room to disinfect one or more surfaces of objects in the room.

5. The disinfection system of claim 2, wherein the controller is configured to change the orientation of the reflector relative to the housing between an occupied position and a plurality of unoccupied positions, wherein the plurality of unoccupied positions span at least 45 degrees.

6. The disinfection system of claim 2, wherein the controller is configured to change the orientation of the reflector relative to the housing between an occupied position and a plurality of unoccupied positions, wherein the plurality of unoccupied positions span at least 90 degrees.

7. The disinfection system of claim 2, wherein the controller is configured to change the orientation of the reflector relative to the housing between an occupied position and a plurality of unoccupied positions, wherein the plurality of unoccupied positions span at least 170 degrees.

8. The disinfection system of claim 1, wherein the source of UV light selectively provides the UV light emission when turned on, and does not provide the UV light emission when turned off.

9. The disinfection system of claim 8, wherein the controller is operatively coupled to the source of UV light and is configured to schedule disinfection of the room at predetermined times, wherein during the predetermined times, the controller is configured to turn on the source of UV light.

10. The disinfection system of claim 1, wherein the source of UV light is configured to provide a plurality of intensities of UV light emission.

11. The disinfection system of claim 10, wherein the controller is operatively coupled to the source of UV light and is configured to control the intensity of the of the UV light emission from the source of UV light.

12. The disinfection system of claim 1, further comprising a plurality of louvers, wherein the reflector directs the UV light emission out of the housing between one or more of the plurality of louvers.

13. A surface and air treatment system comprising:
a source of ultraviolet (UV) light;
a reflector extending partially around the source of UV light;
a motor in communication with the reflector;
an occupancy sensor;
a controller in communication with the source of UV light, the motor, and the occupancy sensor; and
wherein the controller is configured to initiate the source of UV light and cause the motor to adjust a position of the reflector between an air treatment configuration and a surface treatment configuration based on signals from the occupancy sensor.

14. The surface and air treatment system of claim 13, wherein:
the controller is configured to determine whether the occupancy sensor has sensed an occupant in a space adjacent to the source of UV light;
the controller is configured to cause the motor to position the reflector in the surface treatment configuration when the occupancy sensor has not sensed an occupant in the space; and
the controller is configured to cause the motor to maintain the reflector in the air treatment configuration when the occupancy sensor has sensed an occupant in the space.

15. The surface and air treatment system of claim 13, wherein the controller is configured to receive a signal causing the controller to initiate the source of UV light.

16. The surface and air treatment system of claim 15, wherein the signal is received in response to a user manually selecting to initiate the source of UV light.

17. The surface and air treatment system of claim 15, wherein the controller is configured to receive the signal in response to a schedule stored in a remote computing device.

18. The surface and air treatment system of claim 12, wherein the controller is configured to initiate the source of UV light according to a schedule stored in the controller.

19. A method of disinfecting air and surface in a space adjacent a source of disinfectant light, the method comprising:
positioning a reflector extending partially around the source of disinfectant light in an air treatment configuration;
initiating the source of disinfectant light;
determining if an occupant is present in the space adjacent the source of disinfectant light;
when no occupant is present in the space, positioning the reflector to a surface treatment configuration; and
when an occupant is present in the space, maintaining the reflector in the air treatment configuration directing disinfectant light into the space.

20. The method of claim 19, wherein the initiating the source of disinfectant light is in response to one or more of:
a manual user selection to initiate the source of disinfectant light;
a scheduled initiation of the source of disinfectant light; and
a detection of pollutants in the space adjacent the source of disinfectant light.
